(19) United States
(12) Patent Application Publication
(10) Pub. No.: US 2006/0210091 A1
(43) Pub. Date: Sep. 21, 2006

(54) Howling canceler apparatus and sound amplification system

(75) Inventor: Hiraku Okumura, Shizuoka-ken (JP)

Correspondence Address: PILLSBURY WINTHROP SHAW PITTMAN LLP, P.O BOX 10500, McLean, VA, 22102, US

(73) Assignee: Yamaha Corporation, Hamamatsu-shi (JP)

(21) Appl. No.: 11/376,362

(22) Filed: Mar. 15, 2006

(30) Foreign Application Priority Data
Mar. 18, 2005 (JP) 2005-079847

(51) Int. Cl. A61F 11/06; G10K 11/16; H03B 29/00; H04B 3/20

(52) U.S. Cl. 381/71.11; 381/66

(57) ABSTRACT

A howling canceler apparatus is used in a sound amplification system having a sound amplifier which connects with a multiple of speakers and one or more of microphones. In the howling canceler apparatus, a plurality of adaptive filters are provided in correspondence to a plurality of feedback transmission paths which are formed between each of the multiple of the speakers and each of the one or more of the microphones. Each adaptive filter is set with a filter coefficient simulating a transfer function of the corresponding feedback transmission path for processing the output sound signal to generate a simulation signal simulating a feedback sound traveling through the corresponding feedback transmission path. Each adaptive filter is capable of setting its own filter coefficient based on the output sound signal and a residual signal. A subtraction portion subtracts the simulation signal outputted from the adaptive filter from the input sound signal inputted from the microphone to generate the residual signal, and outputs this residual signal to the adaptive filter and to the sound amplifier.

### BACKGROUND OF THE INVENTION

1. Technical Field

The present invention relates to a howling canceler apparatus to prevent howling which is caused by supplying a microphone with feedback sounds from multiple speakers, and also relates to a sound amplification system equipped with this howling canceler apparatus.

2. Related Art

A sound amplification system amplifies a sound signal input from a microphone and inputs the amplified sound signal to a speaker. It is widely known that the sound amplification system forms a closed loop along a path from the speaker to the microphone and howling is generated by repeatedly amplifying a feedback sound signal that is output from the speaker and is input to the microphone.

To prevent such howling, it has been proposed that an adaptive filter is used to generate a simulation signal simulating a feedback sound signal, and the sound amplification system uses a howling canceler apparatus having such an adaptive filter to subtract the simulation signal from an input signal supplied from the microphone (See Inazumi, Imai, and Konishi, “Prevention of acoustic feedback in the sound amplification system using the LMS algorithm,” lecture thesis collection pp. 417-418, The Acoustical Society of Japan, March, 1991). Constituent portions of the howling canceler apparatus operate as follows.

When a sound signal is input to a speaker, the same sound signal as that sound signal is input to a delay portion. The delay portion delays the sound signal for a delay time spent by the sound signal traveling from the speaker to a microphone. A convolution operation is performed for the delayed signal using a filter coefficient of the adaptive filter to generate a simulation signal. A subtraction portion subtracts the simulation signal from the signal input from the microphone to leave a residual signal that is then output to a sound amplification portion. The sound amplification portion amplifies the residual signal that is then input to the speaker. The speaker generates sound. The adaptive filter is supplied with the residual signal as a reference signal. A known adaptive algorithm (e.g., LMS (Least Mean Square) algorithm) is used to update the filter coefficient (filter characteristic) so that the residual signal is minimized. In this manner, the adaptive filter's filter coefficient approximates to a transfer function of the feedback transmission path from the speaker to the microphone. The filter coefficient is used to simulate the feedback transmission path's transfer function. The signal processed by the adaptive filter, i.e., the simulation signal approximates to a feedback sound signal. This makes it possible to remove feedback sound signal components from the input sound signal and prevent the howling.

When multiple speakers are connected, however, a conventional sound amplification system may not be able to stably (statically determinately) simulate transfer functions using the adaptive filter. In this configuration, the sound output from multiple speakers may be input to the same microphone. The same microphone is supplied with feedback sounds transferred by multiple feedback transmission paths. When the same adaptive filter is used to simulate transfer functions for the multiple feedback transmission paths, the transfer functions cannot be simulated stably, making it difficult to accurately prevent the howling.

### SUMMARY OF THE INVENTION

It is therefore an object of the present invention to provide a howling canceler apparatus and a sound amplification system capable of stably simulating transfer functions using adaptive filters and accurately preventing howling even in an acoustic system configuration where multiple feedback paths are formed from speakers to microphones.

To solve the above-mentioned problem, the present invention incorporates the following means.

(1) The present invention provides a howling canceler apparatus included in or connected with a sound amplification system having a sound amplification portion which connects with a multiple of speakers and one or more of microphones and which amplifies an input sound signal inputted from the microphone and supplies the amplified sound signal as an output sound signal to the speakers. The howling canceler apparatus comprises: a plurality of adaptive filters which are provided in correspondence to a plurality of feedback transmission paths which are formed between each of the multiple of the speakers and each of the one or more of the microphones, each adaptive filter being set with a filter coefficient simulating a transfer function of the corresponding feedback transmission path for processing the output sound signal to generate a simulation signal simulating a feedback sound traveling through the corresponding feedback transmission path, each adaptive filter being capable of setting its own filter coefficient based on the output sound signal and a residual signal; and a subtraction portion which subtracts the simulation signal outputted from the adaptive filter from the input sound signal inputted from the microphone to generate the residual signal, and which outputs this residual signal to the adaptive filter and to the sound amplification portion as the input sound signal.

According to the embodiment, the sound amplification system is connected with multiple speakers and one or more microphones. There may be multiple feedback transmission paths between the speakers and the microphones as many as combinations of the speakers and the microphones. That is, there may be feedback transmission paths between the speakers and the microphones for “the number of speakers multiplied by the number of microphones”.

According to the configuration of the present invention, the howling canceler apparatus has the adaptive filter for each of the multiple feedback transmission paths. The adaptive filter sets a filter coefficient based on the output sound signal and the residual signal. The filter coefficient simulates the transfer function for the corresponding feedback transmission path. The adaptive filter is supplied with an output sound signal to be output to the speaker. The adaptive filter processes the output sound signal to generate a simulation signal that simulates the signal associated with the feedback sound supplied from the feedback transmission path. Even when the microphone is supplied with input sound signals via multiple feedback transmission paths, each adaptive filter only needs to simulate the transfer function for one feedback transmission path. This makes it possible to stably simulate the transfer function for the feedback transmission path in comparison with the conventional technology that simulates multiple feedback transmission paths using a single or common adaptive filter.

The subtraction portion subtracts the simulation signal output from the adaptive filter from the input sound signal supplied from the microphone to generate a residual signal. This residual signal is output to the adaptive filter and to the sound amplification portion as the input sound signal. The sound amplification portion can amplify the input sound signal while feedback sound components are fully removed. Accordingly, it is possible to effectively prevent the howling from occurring due to repeated amplification of feedback sound components.

(2) According to the present invention, the above-mentioned howling canceler apparatus is provided with a correlation reduction process portion which decreases correlation among a multiple of the output sound signals, and then feeds these output sound signals after the correlation is decreased to the speakers and the adaptive filters. For example, let us suppose that the speakers generate sounds that acoustically correlate to each other. Even when feedback sound components are input to the microphone via different feedback transmission paths, the feedback sound components may be too highly correlated to be distinguished from each other. In such case, it is difficult to determine which feedback transmission path transmits feedback sound components corresponding to the residual signal input to the adaptive filter. Consequently, it is difficult to stably configure the filter coefficient simulating each feedback transmission path.

According to the above-mentioned embodiment of the present invention, the correlation reduction process portion decreases the correlation among output sound signals output to the multiple speakers. Each of the speakers and adaptive filters is supplied with the output sound signal processed by the correlation reduction process portion. This makes it possible to decrease the correlation among feedback sound components input to the microphone via different feedback transmission paths. Consequently, it is possible to prevent the feedback sound components from being too highly correlated to be distinguished from each other.

(3) According to the present invention, the above-mentioned howling canceler apparatus is provided with another correlation reduction process portion which generates a difference signal by subtracting the output sound signals from each other and a sum signal by adding the output sound signals with each other, wherein the adaptive filter performs a cross spectrum operation using the sum signal and the difference signal to calculate an estimated error between the transfer function of the corresponding feedback transmission path and the simulated transfer function estimated by the adaptive filter itself, and sets the filter coefficient using this estimated error.

According to the above-mentioned configuration of the present invention, the correlation reduction process portion generates a difference signal and a sum signal of output sound signals to be output to the speakers. The speakers are supplied with output sound signals before being processed in the correlation reduction process portion. If the speaker is supplied with the output sound signal processed in the correlation reduction process portion, the speaker may generate a sound whose quality is acoustically degraded. According to the present invention, the speaker is supplied with a signal before being processed in the correlation reduction process portion, making it possible to effectively prevent the acoustic sound quality from being degraded.

On the other hand, the adaptive filter is supplied with a sum signal and a difference signal generated in the correlation reduction process portion. The adaptive filter performs a cross spectrum operation using the sum signal and the difference signal. This operation calculates an estimated error between the transfer function of the corresponding feedback transmission path and the simulated transfer function estimated by the adaptive filter itself. The estimated error is used to calculate the filter coefficient. Accordingly, it is possible to stably set the filter coefficient even when high correlation between sounds generated from the speakers may increase the correlation among feedback sound components input to the microphone via different feedback transmission paths.

(4) In the above-mentioned howling canceler apparatus, according to the present invention, the adaptive filter is supplied with the output sound signal before being processed in the correlation reduction process portion, and convolutes this supplied output sound signal with the filter coefficient to generate the simulation signal.

According to the above-mentioned configuration of the present invention, the adaptive filter convolutes the filter coefficient with the output sound signal before being processed in the correlation reduction process portion. In this manner, the filter coefficient is used to convolute with the sound signal input to each speaker. It is possible to more precisely approximate the simulation signal to the feedback sound than the configuration where the filter coefficient is used to convolute with a sum signal and a difference signal.

(5) Preferably, the inventive howling canceler apparatus further comprises a plurality of delays provided in correspondence to the plurality of the adaptive filters, each delay delaying the output sound signal by a delay time and feeding the delayed output sound signal to the corresponding adaptive filter, the delay time representing a delay time of the feedback sound traveling through the corresponding feedback transmission path.

According to the present invention, the adaptive filter simulates the transfer function for one feedback transmission path even when the microphone is supplied with input sound signals via multiple feedback transmission paths. This makes it possible to provide the sound amplification system simulating each transfer function for each feedback transmission path in comparison with the conventional technology that simulates multiple feedback transmission paths using a common adaptive filter. When the adaptive filter outputs a simulation signal, it is subtracted from the input sound signal. Accordingly, feedback sound components can be fully removed from the input sound signal. It is possible to effectively prevent the howling from occurring.

### BRIEF DESCRIPTION OF THE DRAWINGS

FIG. 1 is a block diagram showing the outline configuration of a sound amplification system according to the first embodiment.

FIG. 2 is a block diagram showing the outline configuration of a sound amplification system according to the second embodiment.

FIG. 3 is a block diagram showing the outline configuration of a sound amplification system according to the third embodiment.

FIG. 4 is a block diagram showing the outline configuration of a sound amplification system according to the fourth embodiment.

### DETAILED DESCRIPTION OF THE INVENTION

Embodiments of the present invention will be described in further detail with reference to the accompanying drawings. In the sound amplification system according to the embodiments, multiple speakers and multiple microphones are connected. Accordingly, the microphones are supplied with a feedback sound output from each of the multiple speakers, i.e., the mixture of multiple feedback sounds fed back through multiple feedback transmission paths. According to the embodiments, a howling canceler apparatus is provided with a delay portion and an adaptive filter corresponding to each of the multiple feedback transmission paths to stably simulate the delay time and the transfer function for each feedback transmission path.

### FIRST EMBODIMENT

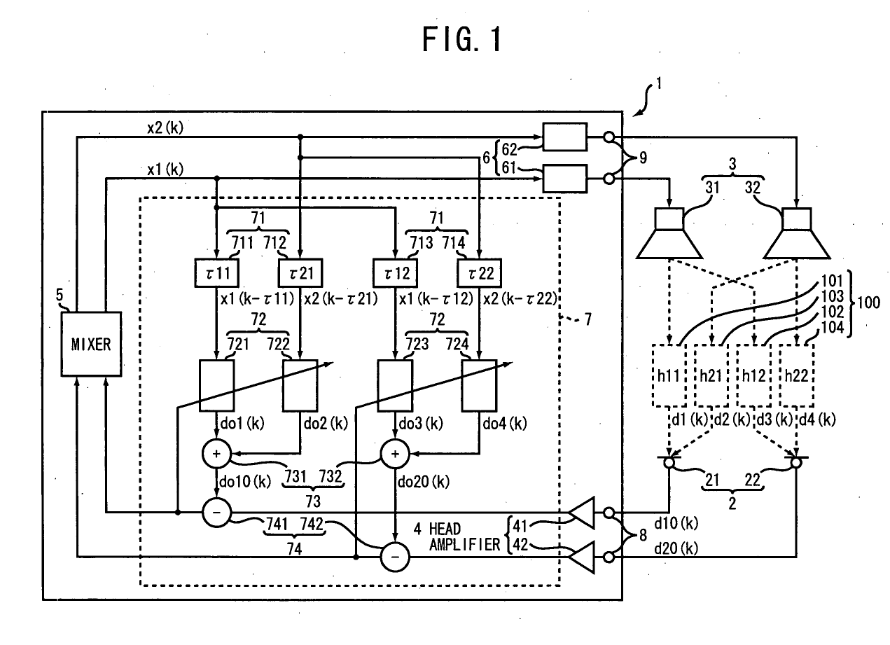

With reference to FIG. 1, the following describes a first embodiment of the present invention. FIG. 1 is a block diagram showing the outline configuration of a sound amplification system 1 according to the first embodiment. The sound amplification system 1 connects with two (multiple) microphones 2 and two (multiple) speakers 3. Each microphone 2 is provided with a head amplifier 4 and a mixer 5. Each speaker 3 is provided with a power amplifier 6 and a howling canceler apparatus 7. The head amplifier 4, mixer 5 and power amplifier 6 may collectively or individually constitute a sound amplification portion of the inventive sound amplification system.

The microphone 2 receives the sound as a microphone input signal from the outside of the apparatus and supplies this microphone input signal to the sound amplification system 1. Of the two microphones 2 in FIG. 1, the left thereof is a microphone 21 the right thereof is a microphone 22. The following description simply denotes the microphone 2 when there is no need for special distinction between the microphones 21 and 22.

The speaker 3 converts the analog sound signal input from the sound amplification system 1 and generates the sound. Of the two speakers in FIG. 1, the left thereof is a speaker 31 that works as a first channel to generate the sound. The right thereof is a speaker 32 that works as a second channel to generate the sound. The following description simply denotes the speaker 3 when there is no need for special distinction between the speakers 31 and 32.

The speakers 31 and 32 and the microphones 21 and 22 are positioned so that the sound generated from the speakers 31 and 32 is input as a feedback sound to each of the microphones 21 and 22 via a feedback transmission path 100 (101, 102, 103, and 104). That is, the sound generated from the speaker 31 is input to not only the microphone 21 via the feedback transmission path 101, but also the microphone 22 via the feedback transmission path 102. The sound generated from the speaker 32 is input to not only the microphone 21 via the feedback transmission path 103, but also the microphone 22 via the feedback transmission path 104. In this manner, the microphone 2 is supplied with the feedback sound via multiple types of the feedback transmission path 100.

The head amplifier 4 (41 and 42) is supplied with the microphone input signal from the microphone 2 via an input terminal 8. The head amplifier 4 amplifies the signal level of the supplied microphone input signal so as to be appropriate to processes for an A/D (Analog/Digital) converter (not shown). The head amplifier 4 inputs the microphone input signal to the A/D converter (not shown). Of the head amplifier 4, a head amplifier 41 is supplied with the microphone input signal from the microphone 21. A head amplifier 42 is supplied with the microphone input signal from the microphone 22. The microphone input signal is amplified in the head amplifiers 41 and 42, digitized in the A/D converter (not shown), and output to a mixer 5.

The mixer 5 mixes and possibly preamplifies input signals. The mixer 5 is supplied with the microphone input signals output from the head amplifiers 41 and 42 via the howling canceler apparatus 7. The mixer mixes these input signals to generate sound signals x1(k) and x2(k). The mixer outputs the sound signal x1(k) to the speaker 31 and outputs the sound signal x2(k) to the speaker 32. The output sound signals x1(k) and x2(k) are input to not only the power amplifier 6, but also the howling canceler apparatus 7. In this manner, the howling canceler apparatus 7 is supplied with the same signals as the sound signals x1(k) and x2(k) input to the speaker 3. According to this configuration, the howling canceler apparatus 7 is supplied with the sound signals x1(k) and x2(k) that do not pass through the power amplifier 6. According to another configuration, the howling canceler apparatus 7 may be supplied with the sound signals x1(k) and x2(k) that pass through the power amplifier 6.

The power amplifier 6 corresponds to the sound amplification portion in the present invention. The power amplifier 6 amplifies signal levels of the input sound signals x1(k) and x2(k) and outputs them to the speaker 3. Two power amplifiers 6 are provided. Of these, a power amplifier 61 outputs signals to the speaker 31. A power amplifier 62 outputs signals to the speaker 32. Signals output from the power amplifiers 61 and 62 are respectively input to the speakers 31 and 32 via an output terminal 9. The power amplifiers 61 and 62 may be digital amplifiers for amplifying digital signals or analog amplifiers for amplifying analog signals. When the analog amplifiers are used, a D/A converter (not shown) is placed previously to the power amplifiers 61 and 62.

The howling canceler apparatus includes a delay portion 71 (711, 712, 713, and 714) an adaptive filter 72 (721, 722, 723, and 724), an addition portion 73 (731 and 732), and a subtraction portion 74 (741 and 742).

The delay portion 71 and the adaptive filter 72 simulates the feedback transmission path 100 that forms a sound transmission route from the speaker 3 to the microphone 2. That is, the delay portion 71 simulates delay time τ of the feedback sound via the feedback transmission path 100. The adaptive filter 72 simulates transfer function h, i.e., the audio propagation characteristic of the feedback transmission path 100. Multiple delay portions 71 and adaptive filters 72 are provided for each of the feedback transmission path 100. That is, the delay portion 711 and the adaptive filter 721 simulate the feedback transmission path 101. The delay portion 712 and the adaptive filter 722 simulate the feedback transmission path 103. The delay portion 713 and the adaptive filter 723 simulate the feedback transmission path 102. The delay portion 714 and the adaptive filter 724 simulate the feedback transmission path 104.

Specifically, the delay portion 71 delays the input sound signals x1(k) and x2(k) for delay time τ that simulates the delay time of the feedback transmission path 100. The delay portion 71 outputs this delayed sound signal x(k-τ) to the adaptive filter 72 that simulates the same feedback transmission path 100 as itself. That is, the delay portion 711 delays sound signal x1(k) for delay time τ11 to simulate the delay time of the feedback transmission path 101 and outputs delayed sound signal x1(k-τ11) to the adaptive filter 721. The delay portion 712 delays sound signal x2(k) for delay time τ21 of the feedback transmission path 103 and outputs delayed sound signal x2(k-τ21) to the adaptive filter 722. The delay portion 713 delays sound signal x1(k) for delay time τ12 of the feedback transmission path 102 and outputs delayed sound signal x1(k-τ12) to the adaptive filter 723. The delay portion 714 delays sound signal x2(k) for delay time τ22 of the feedback transmission path 104 and outputs delayed sound signal x2(k-τ22) to the adaptive filter 724. This specification simply describes delay time “τ” when there is no need for special distinction between delay times τ11, τ21, τ12, and τ22.

The adaptive filter 72 includes a digital filter (typically an FIR (Finite Impulse Response) filter). The adaptive filter 72 estimates transfer function h of the feedback transmission path 100. The adaptive filter 72 calculates this digital filter's filter coefficient (filter characteristic) so as to adjust to (or simulate) the estimated transfer function h and assigns the filter coefficient to itself. The adaptive algorithm is used to estimate transfer function h and calculate the filter coefficient using, as a reference signal, the residual signal output from the subtraction portion 74 based on sound signal x(k-τ) input from the delay portion 71. Applicable adaptive algorithms include the learning identification method, the LMS method, the projection method, and the RLS method, for example. The filter coefficient is calculated at a specified time interval (e.g., every several seconds) so as to generate as small a residual signal as possible. The adaptive filter 72 generates simulation signal do(k) by convoluting the input sound signal x1(k-τ) or x2(k-τ) with the filter coefficient (thus, providing the filter characteristic). The adaptive filter 72 outputs generated simulation signal do(k) to the addition portion 73.

The adaptive filter 721 simulates transfer function h11 for the feedback transmission path 101, generates simulation signal do1(k) by convoluting the input sound signal x1(k-τ11) with the filter coefficient, and outputs generated simulation signal do1(k) to the addition portion 73 (addition portion 731). The adaptive filter 722 simulates transfer function h21 for the feedback transmission path 103, generates simulation signal do2(k) by convoluting the input sound signal x2(k-τ21) with the filter coefficient, and outputs generated simulation signal do2(k) to the addition portion 73 (addition portion 731). The adaptive filter 723 simulates transfer function h12 for the feedback transmission path 102, generates simulation signal do3(k) by convoluting the input sound signal x1(k-τ12) with the filter coefficient, and outputs generated simulation signal do3(k) to the addition portion 73 (addition portion 732). The adaptive filter 724 simulates transfer function h22 for the feedback transmission path 104, generates simulation signal do4(k) by convoluting the input sound signal x2(k-τ22) with the filter coefficient, and outputs generated simulation signal do4(k) to the addition portion 73 (addition portion 732). This specification simply describes simulation signal do(k) when there is no need for special distinction between simulation signals do1(k), do2(k), do3(k), and do4(k).

The addition portion 73 synthesizes simulation signals do(k) with each other. Two (multiple) addition portions 73 are respectively provided for the microphones 21 and 22. The addition portion 731 of the addition portion 73 corresponds to the microphone 21. The addition portion 732 of the addition portion 73 corresponds to the microphone 22. The addition portion 731 is supplied with simulation signals do1(k) and do2(k). The addition portion 731 adds these signals to generate synthesized simulation signal do10(k), thus generating a signal simulating the feedback sound supplied to the microphone 21. The addition portion 732 is supplied with simulation signals do3(k) and do4(k). The addition portion 732 adds these signals to generate synthesized simulation signal do20(k), thus generating a signal simulating the feedback sound supplied to the microphone 22.

The microphone 21 is supplied with synthesized simulation signal d10(k) of feedback sound signals d1(k) and d2(k). The feedback sound d1(k) corresponds to the feedback sound via the feedback transmission path 101. The feedback sound d2(k) corresponds to the feedback sound via the feedback transmission path 103. The microphone 22 is supplied with synthesized simulation signal d20(k) of feedback sound signals d3(k) and d4(k). The feedback sound d3(k) corresponds to the feedback sound via the feedback transmission path 102. The feedback sound d4(k) corresponds to the feedback sound via the feedback transmission path 104. Since the adaptive filter 721 simulates transfer function h11 as mentioned above, simulation signal do1(k) simulates feedback sound signal d1(k). Since the adaptive filter 722 simulates transfer function h21 as mentioned above, simulation signal do2(k) simulates feedback sound signal d1(k). Accordingly, synthesized simulation signal d10(k) approximates to simulation signal do10(k). Since the adaptive filter 723 simulates transfer function h12 as mentioned above, simulation signal do3(k) simulates feedback sound signal d3(k). Since the adaptive filter 724 simulates transfer function h22 as mentioned above, simulation signal do4(k) simulates feedback sound signal d4(k). Accordingly, synthesized simulation signal d20(k) approximates to simulation signal do20(k). This specification simply describes feedback sound signal d(k) when there is no need for special distinction between feedback sound signals d1(k), d2(k), d3(k), and d4(k).

The addition portion 731 inputs generated synthesized simulation signal do10(k) to the subtraction portion 74 (subtraction portion 741 to be described later) corresponding to the microphone 21. The addition portion 732 inputs generated synthesized simulation signal do20(k) to the subtraction portion 74(subtraction portion 742 to be described later) corresponding to the microphone 22. The subtraction portion 74 is supplied with a microphone input signal from the microphone 2. The subtraction portion 74 subtracts synthesized simulation signal do10(k) or do20(k) from the input signal. two subtraction portions 74 are respectively provided for the microphones 21 and 22. The subtraction portion 741 is the subtraction portion 74 corresponding to the microphone 21. The subtraction portion 742 is the subtraction portion 74 corresponding to the microphone 22.

That is, the subtraction portion 741 generates a residual signal by subtracting synthesized simulation signal do10 from the sound signal input from the microphone 21. The subtraction portion 742 generates a residual signal by subtracting synthesized simulation signal do20 from the sound signal input from the microphone 22. The subtraction portion 741 inputs the generated residual signal to the mixer 5 and to the adaptive filters 721 and 722 as the reference signal. The subtraction portion 742 inputs the generated residual signal to the mixer 5 and to the adaptive filters 723 and 724 as the reference signal.

The following describes operations of the sound amplification system 1. When a user speaks, for example, the sound signal such as the user's voice is input to the microphones 21 and 22. The microphone input signal supplied to the microphone 21 is input to the head amplifier 41 via the input terminal 8. The microphone input signal supplied to the microphone 22 is input to the head amplifier 42 via the input terminal 8. The head amplifiers 41 and 42 amplify signal levels of the supplied microphone input signals. The microphone input signals are then input to the mixer 5 via the subtraction portions 741 and 742. The mixer 5 mixes the microphone input signals supplied from the microphones 21 and 22 to generate sound signals x1(k) and x2(k).

The mixer inputs the generated sound signals x1(k) and x2(k) not only to the power amplifiers 61 and 62, but also to the delay portions 711, 712, 713, and 714. That is, sound signal x1(k) input to the power amplifier 61 is also input to the delay portions 711 and 713. Sound signal x2(k) input to the power amplifier 62 is also input to the delay portions 712 and 714. The power amplifiers 61 and 62 amplify signal levels of the input sound signals x1(k) and x2(k) that are then input to the speakers 31 and 32 via the output terminal 9.

The analog signal input to the speaker 31 is transformed into sound that is then generated audibly. The sound is input as feedback sound signal d1(k) to the microphone 21 via the feedback transmission path 101. The sound is also input as feedback sound signal d3(k) to the microphone 22 via the feedback transmission path 102. The analog signal input to the speaker 32 is transformed into sound that is then generated audibly. The sound is input as feedback sound signal d2(k) to the microphone 21 via the feedback transmission path 103. The sound is also input as feedback sound signal d4(k) to the microphone 22 via the feedback transmission path 104. That is, the microphone 21 is supplied with synthesized simulation signal d10(k) composed of feedback sound signals d1(k) and d2(k). The microphone 22 is supplied with synthesized simulation signal d20(k) composed of feedback sound signals d3(k) and d4(k).

The howling canceler apparatus 7 uses the delay portions 711, 712, 713, and 714 to provide delay time τ for sound signals x1(k) and x2(k). That is, the delay portion 711 provides delay time τ11 to sound signal x1(k) to generate sound signal x1(k-τ11) that is then input to the adaptive filter 721. The delay portion 712 provides delay time τ21 to sound signal x2(k) to generate sound signal x2(k-τ2l) that is then input to the adaptive filter 722. The delay portion 713 provides delay time τ12 to sound signal x1(k) to generate sound signal x1(k-τ12) that is then input to the adaptive filter 723. The delay portion 714 provides delay time τ22 to sound signal x2(k) to generate sound signal x2(k-τ22) that is then input to the adaptive filter 724.

The adaptive filter 721 supplies sound signal x1(k-τ11) with the filter characteristic corresponding to the feedback transmission path 101 to generate simulation signal do1(k). The generated simulation signal do1(k) is input to the addition portion 731. The adaptive filter 722 supplies sound signal x2(k-τ21) with the filter characteristic corresponding to the feedback transmission path 103 to generate simulation signal do2(k). The generated simulation signal do2(k) is input to the addition portion 731. The adaptive filter 723 supplies sound signal x1(k-τ12) with the filter characteristic corresponding to the feedback transmission path 102 to generate simulation signal do3(k). The generated simulation signal do3(k) is input to the addition portion 732. The adaptive filter 724 supplies sound signal x2(k-τ22) with the filter characteristic corresponding to the feedback transmission path 104 to generate simulation signal do4(k). The generated simulation signal do4(k) is input to the addition portion 732.

The addition portion 731 adds simulation signals do1(k) and do2(k) to generate synthesized simulation signal do10(k). The synthesized simulation signal do10(k) is input to the subtraction portion 741. The addition portion 732 adds simulation signals do3(k) and do4(k) to generate synthesized simulation signal do20(k). The synthesized simulation signal do20(k) is input to the subtraction portion 742. The subtraction portion 742 removes synthesized simulation signal do10(k) from the microphone input signal supplied from the microphone 21 to remove components of synthesized simulation signal d10(k). The subtraction portion 742 removes synthesized simulation signal do20(k) from the microphone input signal supplied from the microphone 22 to remove components of synthesized simulation signal d20(k). This method removes feedback sound components supplied from microphone input signals supplied from the microphones 21 and 22 via multiple feedback transmission paths 100. It is possible to effectively prevent the howling.

According to the above-mentioned configuration, the embodiment provides multiple types of adaptive filters 72 even when the same microphone 2 is supplied with the feedback sound via multiple types of feedback transmission paths 100. In this manner, the delay time is supplied for each feedback transmission path 100 and transfer function h is simulated. It is possible to stably estimate transfer function h. As a result, synthesized simulation signals do10(k) and do20(k) can be accurately approximated to synthesized simulation signals d10(k) and d20(k). It is possible to accurately prevent the howling.

Further, the delay portion 71 is provided for each feedback transmission path 100. Sound signal x(k) is delayed for delay time τ corresponding to each feedback transmission path 100 and is input to the adaptive filter 72. It is possible to accurately match the input timing between feedback sound signal d(k) and simulation signal do(k) supplied to the subtraction portion 74. Since simulation signal do(k) is removed from the simulation signal, it is possible to appropriately remove feedback sound components corresponding to simulation signal do(k). Accordingly, this makes it possible to accurately prevent the howling.

### SECOND EMBODIMENT

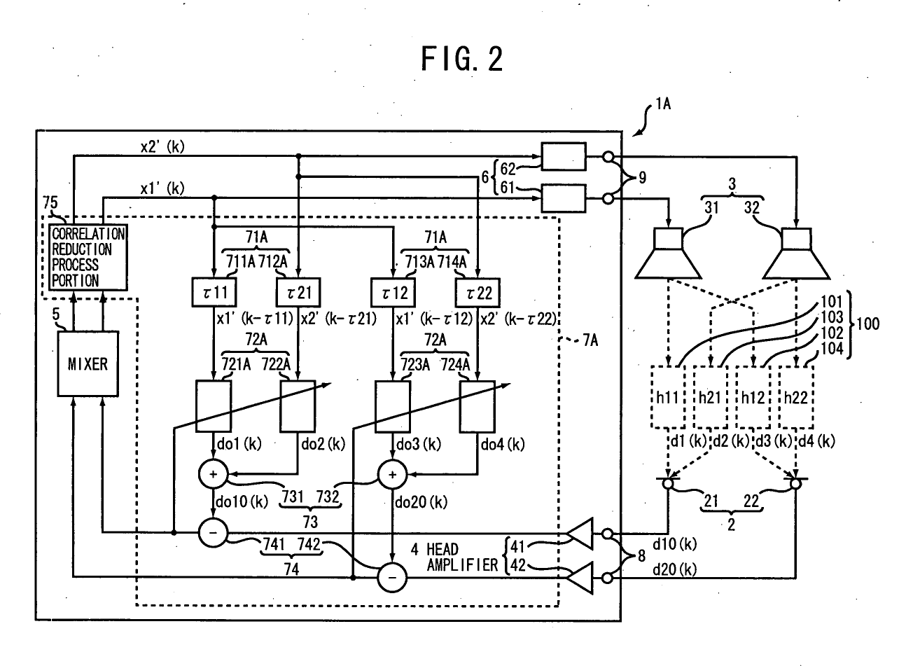

Referring now to FIG. 2, the following describes a sound amplification system 1A according to a second embodiment of the present invention. FIG. 2 is a block diagram showing the outline configuration of the sound amplification system 1A according to the second embodiment of the present invention. According to the first embodiment, the speakers 31 and 32 are supplied with sound signals x1(k) and x2(k) supplied from the mixer 5 via the power amplifier 6. The delay portion 71 is supplied with sound signals x1(k) and x2(k) output from the mixer 5. By contrast, the second embodiment performs a process (correlation reduction process) to decrease the correlation between sound signals x1(k) and x2(k). After this process, sound signals x1′(k) and x2′(k) are respectively input to the speakers 31 and 32 via the power amplifier 6 and also to delay portions 711A and 713A and 712A and 714A.

In addition to the same configuration as the howling canceler apparatus 7, the howling canceler apparatus 7A in FIG. 2 is provided with a correlation reduction process portion 75. The correlation reduction process portion 75 is positioned along the signal route between the mixer 5 and the power amplifier 6 and between the mixer 5 and a branch to the delay portion 71A on this signal route. The correlation reduction process portion 75 is equivalent to a first correlation reduction process portion according to the present invention. The correlation reduction process portion 75 applies a correlation reduction process to sound signals x1(k) and x2(k) supplied from the mixer 5. The correlation reduction process portion 75 applies the correlation reduction process to sound signal x1(k) to generate sound signal x1′(k) and supplies sound signal x1′(k) to the power amplifier 61 and the delay portions 711A and 713A. The correlation reduction process portion 75 applies the correlation reduction process to sound signal x2(k) to generate sound signal x2′(k) and supplies sound signal x2′(k) to the power amplifier 62 and the delay portions 712A and 714A.

The correlation reduction process portion 75 performs the following correlation reduction processes, for example. One process supplies one of sound signals x1(k) and x2(k) with noise components such as white noise as an identification signal. Another process (MS system) generates a sum signal and a difference signal between sound signals x1(k) and x2(k) and uses them as sound signals x1′(k) and x2′(k), respectively. Yet another process (orthogonalization) analyzes main components of sound signals x1(k) and x2(k) and transforms these signals into two signals that are orthogonal to each other.

Similarly to the first embodiment, each delay portion 71A delays input sound signals x1′(k) and x2′(k) for delay time τ that corresponds to the delay time for each feedback transmission path 100. In this manner, the delay portion 71A generates sound signals x1′(k-τ) and x2′(k-τ) and supplies these signals to an adaptive filter 72A. The adaptive filter 72A convolutes the input sound signals x1′(k-τ) and x2′(k-τ) with the filter coefficient to generate simulation signal do(k). Similarly to the first embodiment, the adaptive filter 72A supplies simulation signal do(k) to the addition portions 731 and 732. The signal processes in the addition portion 73 and the subtraction portion 74 are the same as those in the first embodiment and a description is omitted.

The adaptive filter 72A uses the supplied sound signals x1′(k-τ) and x2′(k-τ) and the residual signal to calculate the filter coefficient using the adaptive algorithm similarly to the first embodiment. The calculated filter coefficient is used for correction. That is, an adaptive filter 721A calculates the filter coefficient using supplied sound signal x1(k-τ11) and the residual signal supplied from the subtraction portion 741. An adaptive filter 722A calculates the filter coefficient using supplied sound signal x2′(k-τ21) and the residual signal supplied from the subtraction portion 741. An adaptive filter 723A calculates the filter coefficient using supplied sound signal x1′(k-τ12) and the residual signal supplied from the subtraction portion 742. An adaptive filter 724A calculates the filter coefficient using supplied sound signal x2′(k-τ22) and the residual signal supplied from the subtraction portion 742.

When there is close correlation between sounds generated from the speakers 31 and 32, for example, the correlation increases between feedback sound signals d1(k) and d2(k) input to the microphone 21. The correlation also increases between feedback sound signals d3(k) and d4(k) input to the microphone 22. For this reason, it is difficult to determine whether the residual signal originates from feedback sound signal d1(k) or d2(k). Further, it is difficult to determine whether the residual signal originates from feedback sound signal d3(k) or d4(k). The second embodiment prevents this situation as follows. The correlation reduction process portion 75 applies the correlation reduction process to mixed sound signals x1(k) and x2(k) to decrease the correlation between them. The sound signals are supplied as x1′(k) and x2′(k) to the speakers 31 and 32.

According to the above-mentioned configuration, the second embodiment uses the correlation reduction process portion 75 to supply the speakers 31 and 32 with sound signals x1′(k) and x2′(k) whose correlation is decreased. It is possible to effectively prevent the difficulty in determining whether the residual signal originates from feedback sound components transmitted to which feedback transmission path 100. An appropriate filter coefficient can be calculated.

### THIRD EMBODIMENT

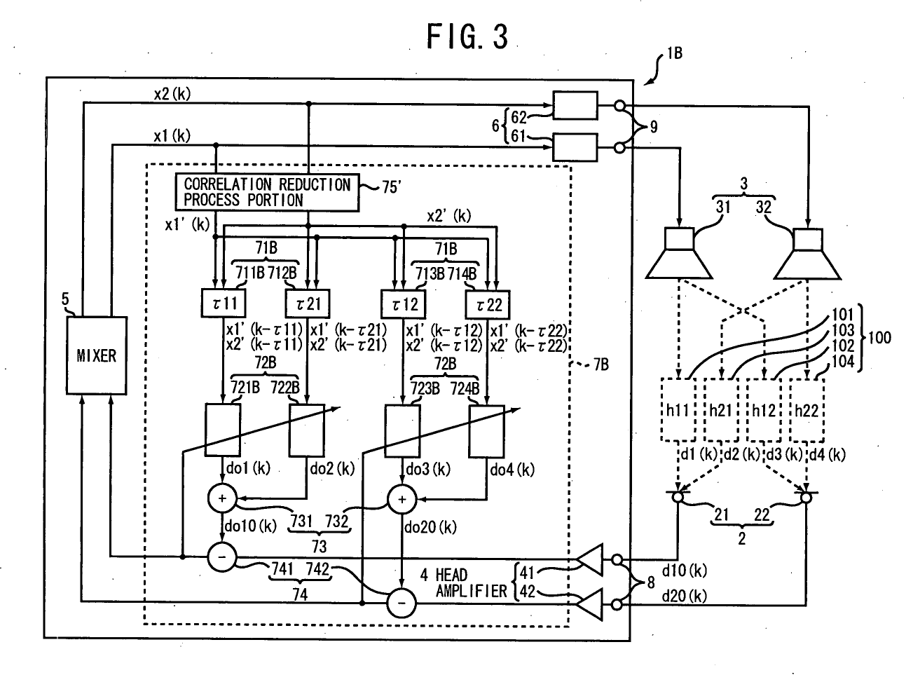

Referring now to FIG. 3, the following describes a third embodiment of the present invention. FIG. 3 is a block diagram showing the outline configuration of a sound amplification system 1B according to the third embodiment of the present invention. According to the second embodiment, the correlation reduction process portion 75 supplies the delay portion 71A and the speakers 31 and 32 with sound signals x1′(k) and x2′(k) to which the correlation reduction process is applied. This configuration decreases the correlation between sounds generated from the speakers 31 and 32. In this manner, it is possible to use the adaptive filter 72A to stably calculate the filter coefficient. By contrast, the third embodiment supplies the speakers 31 and 32 with sound signals x1(k) and x2(k) to which no correlation reduction process is applied. This does not decrease the correlation between sounds generated from the speakers 31 and 32. To solve this problem, a correlation reduction process portion 75′ supplies a delay portion 71B with sound signals x1′(k) and x2′(k) to which the correlation reduction process is applied. Each adaptive filter 72B performs an estimated error calculation process (to be described) using sound signals x1′(k) and x2′(k) and the residual signal to calculate estimated error Δh between transfer function h for the feedback transmission path 100 and the transfer function estimated by the adaptive filter 72B itself. The adaptive filter 72B uses this estimated error Δh to calculate the filter coefficient. Since each adaptive filter 72B calculates the filter coefficient using estimated error Δh, the filter coefficient can be stably calculated. In this manner, the third embodiment is characterized by stably calculating the filter coefficient while maintaining the quality of generated sound.

In the sound amplification system 1B of FIG. 3, the correlation reduction process portion 75′ is positioned along the signal route between the delay portion 71B and the branch from the signal route between the mixer 5 and the power amplifier 6. The correlation reduction process portion 75′ uses the MS system as mentioned in the second embodiment to apply the correlation reduction process to sound signals x1(k) and x2(k) supplied from the mixer 5. The processed sound signals are input to the delay portion 71B.

Specifically, the correlation reduction process portion 75′ is composed of a subtractor, an adder, and the like. The MS-based correlation reduction process generates a sum signal (sound signal x1′(k)) of sound signals x1(k) and x2(k) and a difference signal (sound signal x2′(k)) between sound signals x1(k) and x2(k), i.e., “x1(k)-x2(k)” or “x2(k)-x1(k)”. The correlation reduction process portion 75′ supplies sound signals x1′(k) and x2′(k) to the delay portions 711B, 712B, 713B, and 714B.

The delay portion 711B delays sound signals x1′(k) and x2′(k) supplied using delay time τ11 corresponding to the delay time for each feedback transmission path 100 similarly to the first embodiment to generate sound signals x1′(k-τ11) and x2′(k-τ11) that are then input to the adaptive filter 721B. The delay portion 712B delays sound signals x1′(k) and x2′(k) supplied using delay time τ21 corresponding to the delay time for each feedback transmission path 100 similarly to the first embodiment to generate sound signals x1′(k-τ21) and x2′(k-τ21) that are then input to the adaptive filter 722B. The delay portion 713B delays sound signals x1′(k) and x2′(k) supplied using delay time τ12 corresponding to the delay time for each feedback transmission path 100 similarly to the first embodiment to generate sound signals x1′(k-τ12) and x2′(k-τ12) that are then input to the adaptive filter 723B. The delay portion 714B delays sound signals x1′(k) and x2′(k) supplied using delay time τ22 corresponding to the delay time for each feedback transmission path 100 similarly to the first embodiment to generate sound signals x1′(k-τ22) and x2′(k-τ22) that are then input to the adaptive filter 724B.

Each adaptive filter 72B convolutes the supplied sound signal x1′(k-τ) or k2′(k-τ) with the filter coefficient to generate simulation signal do(k). Specifically, the adaptive filter 721B convolutes the supplied x1′(k-τ11) with the filter coefficient to generate simulation signal do1(k) and supplies it to the addition portion 731 similarly to the first embodiment. The adaptive filter 722B convolutes the supplied x2′(k-τ21) with the filter coefficient to generate simulation signal do2(k) and supplies it to the addition portion 731 similarly to the first embodiment. The adaptive filter 723B convolutes the supplied x1′(k-τ12) with the filter coefficient to generate simulation signal do3(k) and supplies it to the addition portion 732 similarly to the first embodiment. The adaptive filter 724B convolutes the supplied x2′(k-τ22) with the filter coefficient to generate simulation signal do4(k) and supplies it to the addition portion 732 similarly to the first embodiment.

Each adaptive filter 72B performs a cross spectrum operation using the supplied sound signals x1′(k-τ) and x2′(k-τ) and the residual signal to calculate estimated error Δh between the transfer function simulated by each adaptive filter 72B and transfer function h for the corresponding feedback transmission path 100. Each adaptive filter 72B uses the calculated estimated error Δh to calculate the filter coefficient and assigns the calculated filter coefficient to itself.

Specifically, the adaptive filter 721B uses sound signals x1′(k-τ11) and x2′(k-τ11) and the residual signal supplied from the subtraction portion 741. The adaptive filter 721B further uses the following equation to calculate estimated error Δh11 and uses this estimated error Δh11 to calculate the filter coefficient.
Estimated error Δh11=ΣX1′*×E_{L}/Σ|X1′|²+ΣX2′*×E_{L}/Σ|X2′|² [Equation 1]

In this equation, X1′ represents sound signals x1′(k-τ11), x1′(k-τ21), x1′(k-τ12), and x1′(k-τ22) in terms of the frequency axis. X2′ represents x2′(k-τ11), x2′(k-τ21), x2′(k-τ12), and x2′(k-τ22) in terms of the frequency axis. X1′* is the complex conjugate of X1′ and X2′* is the complex conjugate of X2′. E_{L} represents the residual signal supplied from the subtraction portion 741 in terms of the frequency axis.

The adaptive filter 722B uses sound signals x1′(k-τ21) and x2′(k-τ21) and the residual signal supplied from the subtraction portion 741. The adaptive filter 722B further uses the following equation to calculate estimated error Δh21 and uses this estimated error Δh21 to calculate the filter coefficient.
Estimated error Δh21 ΣX1′*×E_{L}/Σ|X1′|²−ΣX2′*×E_{L}/Σ|X2′|² [Equation 2]

Specifically, the adaptive filter 723B uses sound signals x1′(k-τ12) and x2′(k-τ12) and the residual signal supplied from the subtraction portion 742. The adaptive filter 723B further uses the following equation to calculate estimated error Δh12 and uses this estimated error Δh12 to calculate the filter coefficient.
Estimated error Δh12=ΣX1′*×E_{R}/Σ|X1′|²+ΣX2′*×E_{R}/Σ|X2′|² [Equation 3]

In this equation, E_{R} represents the residual signal supplied from the subtraction portion 742 in terms of the frequency axis.

The adaptive filter 724B uses sound signals x1′(k-τ22) and x2′(k-τ22) and the residual signal supplied from the subtraction portion 742. The adaptive filter 724B further uses the following equation (4) to calculate estimated error Δh22 and uses this estimated error Δh22 to calculate the filter coefficient.
Estimated error Δh22=ΣX1′*×E_{R}/Σ|X1′|²−ΣX2′*×E_{R}/Σ|X2′|² [Equation 4]

As disclosed in Japanese Non-examined Patent Publication No. 2003-102085, for example, the known method is used to calculate the filter coefficient using estimated errors Δh11, 12, 21, and 22, and a description is omitted.

According to the above-mentioned configuration, the third embodiment performs the cross spectrum operation using the residual signal and sound signals x1′(k-τ) and x2′(k-τ) to which the correlation reduction process portion 75 applies the correlation reduction process. Consequently, it is possible to calculate estimated error Δh between each adaptive filter 72B and the transfer function for the corresponding feedback transmission path. Estimated error Δh can be used to calculate the filter coefficient for each adaptive filter 72B. Even when the speakers 31 and 32 generate highly correlated sounds, the filter coefficient can be stably calculated. When the speakers 31 and 32 are supplied with sound signals x1(k) and x2(k) to which no correlation reduction process is applied, the filter coefficient for the adaptive filter 72B can be stably calculated. Compared to the second embodiment that supplies the speakers 31 and 32 with sound signals x1′(k) and x2′(k) to which the correlation reduction process is applied, it is possible to prevent deterioration of the quality of sounds generated from the speakers 31 and 32. In addition, the filter coefficient can be stably calculated.

The present invention is not limited thereto and may apply the correlation reduction process according to the orthogonal transform as mentioned above in the second embodiment. According to the modification, the correlation reduction process portion 75′ is composed of an orthogonalization filter and the like. The correlation reduction process portion 75′ analyzes main components of sound signals x1(k) and x2(k) at a specified time interval and transforms sound signals x1(k) and x2(k) into two signals that are orthogonal to each other (having phases shifted 90 degrees). The correlation reduction process portion 75′ supplies sound signals x1′(k) and x2′(k) to delay portions 711B, 712B, 713B, and 714B. Similarly to the third embodiment, the delay portion 71B provides delay time τ for the supplied sound signals x1′(k) and x2′(k) and supplies these signals to the adaptive filter 72B. The adaptive filters 721B and 723B convolute sound signal x1′(k-τ) with the filter coefficient to generate simulation signals do1(k) and do3(k). The adaptive filters 722B and 724B convolute sound signal x2′(k-τ) with the filter coefficient to generate simulation signals do2(k) and do4(k). Each adaptive filter 72B calculates estimated error Δh for the transfer function using sound signals x1′(k-τ) and x2′(k-τ) and the residual signal. The specific calculation method complies with the publicly know technology as disclosed in Japanese Non-examined Patent Publication No. 2003-102085, for example, and a description is omitted. The other configurations and signal processes in this modification are the same as those described in the third embodiment and a description is omitted.

### FOURTH EMBODIMENT

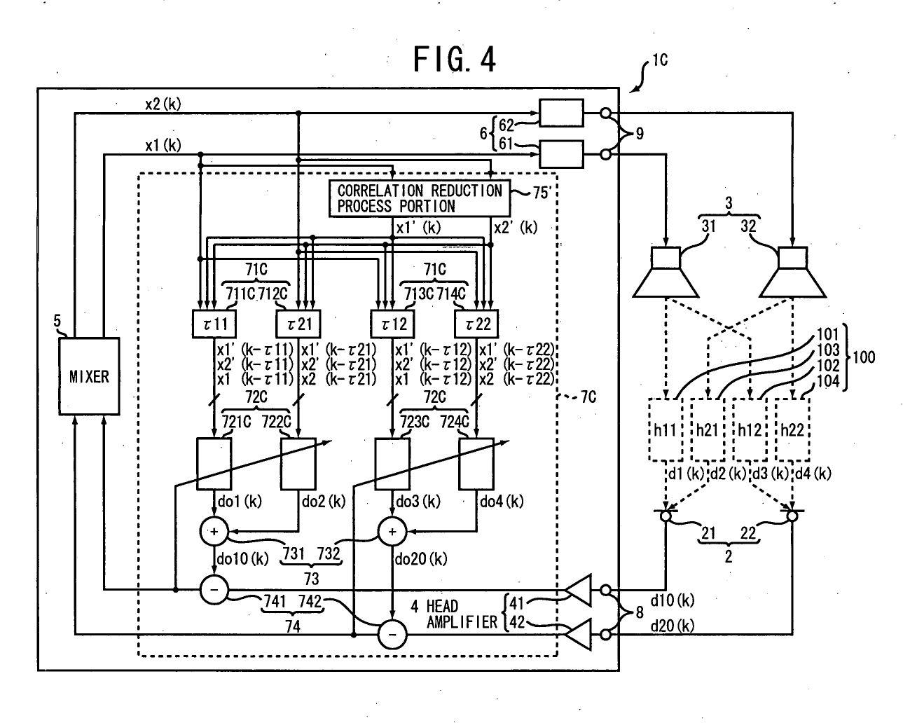

Referring now to FIG. 4, the following describes a sound amplification system 1C according to a fourth embodiment of the present invention. FIG. 4 is a block diagram showing the outline configuration of the sound amplification system 1C according to the fourth embodiment of the present invention. According to the third embodiment, each adaptive filter 72B uses the filter coefficient to perform the convolution operation for sound signal x1′(k-τ) or x2′(k-τ), i.e., sound signals to which the correlation reduction process is applied. According to the fourth embodiment, each adaptive filter 72C uses the filter coefficient to perform the convolution operation for sound signal x1(k-τ) or x2(k-τ).

The delay portion 75′ supplies the delay portion 71C with not only sound signals x1′(k) and x2′(k), but also sound signal x1(k) or x2(k). That is, sound signal x1(k) is supplied to the delay portions 711C and 713C. Sound signal x2(k) is supplied to the delay portions 712C and 714C. The delay portion 711C delays supplied sound signals x1′(k), x2′(k), and x1(k) for delay time τ11 and supplies these signals to the adaptive filter 721C. The delay portion 712C delays supplied sound signals x1′(k), x2′(k), and x2(k) for delay time τ21 and supplies these signals to the adaptive filter 722C. The delay portion 713C delays supplied sound signals x1′(k), x2′(k), and x1(k) for delay time τ12 and supplies these signals to the adaptive filter 723C. The delay portion 714C delays supplied sound signals x1′(k), x2′(k), and x2(k) for delay time τ22 and supplies these signals to the adaptive filter 724C.

Similarly to the third embodiment, the adaptive filter 72C calculates the filter coefficient using the supplied sound signals x1′(k-τ) and x2′(k-τ) and the residual signal. The adaptive filter 72C assigns the calculated filter coefficient to itself. The adaptive filter 72C generates simulation signal do(k) by convoluting the supplied sound signal x1(k-τ) or x2(k-τ) with the filter coefficient. Specifically, the adaptive filter 721C convolutes sound signal x1(k-τ11) with the filter coefficient to generate simulation signal do1(k) and supplies it to the addition portion 731. The adaptive filter 722C convolutes sound signal x2(k-τ21) with the filter coefficient to generate simulation signal do2(k) and supplies it to the addition portion 731. The adaptive filter 723C convolutes sound signal x1(k-τ12) with the filter coefficient to generate simulation signal do3(k) and supplies it to the addition portion 732. The adaptive filter 724C convolutes sound signal x2(k-τ22) with the filter coefficient to generate simulation signal do4(k) and supplies it to the addition portion 732. The other configurations and signal processes of the sound amplification system 1C are the same as those described in the third embodiment and a description is omitted.

According to the above-mentioned configuration, the fourth embodiment delays sound signals x1(k) and x2(k) identical to those supplied to the speakers 31 and 32 to generate sound signals x1(k-τ) and x2(k-τ). The fourth embodiment can convolute these delayed signals with the filter coefficient to generate simulation signal do(k). It is possible to more accurately generate simulation signal do(k) approximate to feedback sound signal d(k). This makes it possible to further improve the accuracy of preventing the howling.

The embodiments of the present invention can employ the following modifications.

(1) According to the first through fourth embodiments, the sound amplification systems 1, 1A, 1B, and 1C are configured to be attached with the microphone 2 and the speaker 3 externally. The present invention is not limited thereto. The sound amplification systems 1, 1A, 1B, and 1C may be integrated with the microphone 2 and the speaker 3. The sound amplification systems 1, 1A, 1B, and 1C include the howling canceler apparatuses 7, 7A, 7B, and 7B but may connect with these howling canceler apparatuses externally.

(2) According to the first through fourth embodiments, the sound amplification systems 1, 1A, 1B, and 1C connect with the two microphones 2 and the two speakers 3. The present invention is not limited thereto. The embodiments only need to connect with the multiple speakers 3 and supply at least one microphone 2 with feedback sounds from the multiple feedback transmission paths 100. The single microphone 2 may be provided. In this case, the adaptive filters 72, 72A, 72B, and 72C are provided for the number of feedback transmission paths 100. When one microphone 2 and the two speakers 3 are connected, the microphone 2 is normally supplied with feedback sounds via the two feedback transmission paths 100. Accordingly, there are provided two adaptive filters 72, 72A, 72B, and 72C corresponding to the two feedback transmission paths 100.

(3) There may be a case where the speaker 3 is distant from the microphone 2 too far to transmit the feedback sound. In such case, it is assumed that there is no feedback transmission path 100. It may be unnecessary to provide the corresponding adaptive filters 72, 72A, 72B, and 72C. With respect to the first embodiment, for example, let us assume that the speaker 31 is distant from the microphone 21 too far to transmit the feedback sound. Since it is assumed that there is no feedback transmission path 101, the delay portion 711 and the adaptive filter 721 are unneeded.

(4) The second embodiment provides the correlation reduction process portion 75 independently of the mixer 5. Further or alternatively, the mixer 5 may have the function of the correlation reduction process portion 75.

(5) According to the third and fourth embodiments, the correlation reduction process portion 75′ is provided along the signal route from an intermediate branch along the signal route between the mixer 5 and the power amplifier 6. The present invention is not limited to this configuration. The third embodiment only needs to be configured so that the speakers 31 and 32 can be supplied with sound signals x1(k) and x2(k), and that the delay portion 71B can be supplied with sound signals x1′(k) and x2′(k) (sound signals applied with the correlation reduction process). The fourth embodiment only needs to be configured so that the speakers 31 and 32 can be supplied with sound signals x1(k) and x2(k), and that the delay portion 71C can be supplied with not only sound signals x1′(k) and x2′(k) (sound signals applied with the correlation reduction process), but also sound signals x1(k) and x2(k). For example, the correlation reduction process portion 75′ may be provided at a connection position similar to the correlation reduction process portion 75 according to the second embodiment. The power amplifier 6 may be preceded by a processing portion that retransforms sound signals x1′(k) and x2′(k) to x1(k) and x2(k). For example, this processing portion halves (sound signal x1′(k) +sound signal x2′(k)) to find sound signal x1(k). The processing portion halves (sound signal x1′(k)−sound signal x2′(k)) to find sound signal x2(k).

1. A howling canceler apparatus included in or connected with a sound amplification system having a sound amplification portion which connects with a multiple of speakers and one or more of microphones and which amplifies an input sound signal inputted from the microphone and supplies the amplified sound signal as an output sound signal to the speakers, the howling canceler apparatus comprising:
a plurality of adaptive filters which are provided in correspondence to a plurality of feedback transmission paths which are formed between each of the multiple of the speakers and each of the one or more of the microphones, each adaptive filter being set with a filter coefficient simulating a transfer function of the corresponding feedback transmission path for processing the output sound signal to generate a simulation signal simulating a feedback sound traveling through the corresponding feedback transmission path, each adaptive filter being capable of setting its own filter coefficient based on the output sound signal and a residual signal; and
a subtraction portion which subtracts the simulation signal outputted from the adaptive filter from the input sound signal inputted from the microphone to generate the residual signal, and which outputs this residual signal to the adaptive filter and to the sound amplification portion as the input sound signal.

2. The howling canceler apparatus according to claim 1, further comprising:
a correlation reduction process portion which decreases correlation among a multiple of the output sound signals, and then feeds these output sound signals after the correlation is decreased to the speakers and the adaptive filters.

3. The howling canceler apparatus according to claim 1, further comprising:
a correlation reduction process portion which generates a difference signal by subtracting the output sound signals from each other and a sum signal by adding the output sound signals with each other,
wherein the adaptive filter performs a cross spectrum operation using the sum signal and the difference signal to calculate an estimated error between the transfer function of the corresponding feedback transmission path and the simulated transfer function estimated by the adaptive filter itself, and sets the filter coefficient using this estimated error.

4. The howling canceler apparatus according to claim 3,
wherein the adaptive filter is supplied with the output sound signal before being processed in the correlation reduction process portion, and convolutes this supplied output sound signal with the filter coefficient to generate the simulation signal.

5. The howling canceler apparatus according to claim 1, further comprising a plurality of delays provided in correspondence to the plurality of the adaptive filters, each delay delaying the output sound signal by a delay time and feeding the delayed output sound signal to the corresponding adaptive filter, the delay time representing a delay time of the feedback sound traveling through the corresponding feedback transmission path.

6. A sound amplification system comprising:
a multiple of speakers and one or more of microphones, which are arranged to form a plurality of feedback transmission paths between each of the multiple of the speakers and each of the one or more of the microphones;
a sound amplification portion which connects between the multiple of the speakers and the one or more of the microphones and which amplifies an input sound signal inputted from the microphone and supplies the amplified sound signal as an output sound signal to the speakers;
a plurality of adaptive filters which are provided in correspondence to the plurality of the feedback transmission paths, each adaptive filter being set with a filter coefficient simulating a transfer function of the corresponding feedback transmission path for processing the output sound signal to generate a simulation signal simulating a feedback sound traveling through the corresponding feedback transmission path, each adaptive filter being capable of setting its own filter coefficient based on the output sound signal and a residual signal; and
a subtraction portion which subtracts the simulation signal outputted from the adaptive filter from the input sound signal inputted from the microphone to generate the residual signal, and which outputs this residual signal to the adaptive filter and to the sound amplification portion as the input sound signal.

7. The sound amplification system according to claim 6, further comprising:
a correlation reduction process portion which decreases correlation among a multiple of the output sound signals, and then feeds these output sound signals after the correlation is decreased to the speakers and the adaptive filters.

8. The sound amplification system according to claim 6, further comprising:
a correlation reduction process portion which generates a difference signal by subtracting the output sound signals from each other and a sum signal by adding the output sound signals with each other,
wherein the adaptive filter performs a cross spectrum operation using the sum signal and the difference signal to calculate an estimated error between the transfer function of the corresponding feedback transmission path and the simulated transfer function estimated by the adaptive filter itself, and sets the filter coefficient using this estimated error.

9. The sound amplification system according to claim 8,
wherein the adaptive filter is supplied with the output sound signal before being processed in the correlation reduction process portion, and convolutes this supplied output sound signal with the filter coefficient to generate the simulation signal.

10. The sound amplification system according to claim 6, further comprising a plurality of delays provided in correspondence to the plurality of the adaptive filters, each delay delaying the output sound signal by a delay time and feeding the delayed output sound signal to the corresponding adaptive filter, the delay time representing a delay time of the feedback sound traveling through the corresponding feedback transmission path.
